(19) United States
(12) Patent Application Publication
(10) Pub. No.: US 2022/0241508 A1
(43) Pub. Date: Aug. 4, 2022

(54) MEDICAL HOLLOW ARTICLE, MEDICAL HOLLOW ARTICLE HAVING A CLOSURE CAP, METHOD FOR PRODUCING A MEDICAL HOLLOW ARTICLE, AND KIT

(71) Applicant: Vetter Pharma-Fertigung GmbH & Co. KG, Ravensburg (DE)

(72) Inventor: Joachim Glocker, Ravensburg (DE)

(21) Appl. No.: 17/612,489

(22) PCT Filed: May 26, 2020
(86) PCT No.: PCT/EP2020/064615

§ 371 Date: Nov. 18, 2021

(30) Foreign Application Priority Data
May 29, 2019 (DE) 10 2019 207 970.9

(51) Int. Cl. A61M 5/28; A61J 1/06; A61J 1/14; A61J 1/20; A61M 5/31

(52) U.S. Cl. CPC A61M 5/28; A61J 1/062; A61J 1/1425; A61M 2207/00; A61M 5/3134; A61M 2005/3104; A61M 2005/3106; A61J 1/2096

(57) ABSTRACT

The invention relates to a medical hollow container (1) made of glass, the medical hollow container (1) having a fastening portion (3) and a receiving portion (5), the fastening portion (3) having a shoulder region (7) and having a head region (9), and the head region (9) having an opening (11) on which a closure cap (13) can be arranged. It is provided that the fastening portion (3) has at least one rotation inhibiting structure (15) which is designed to at least inhibit relative rotation between the medical hollow container (1) and the closure cap (13) with respect to each other, the rotation inhibiting structure (15) having at least one rotation inhibiting element (17), the at least one rotation inhibiting element (17) being at least one elevation and/or at least one depression, and/or being designed as a non-circular shape of the opening (11) and/or a non-circular edge of the opening (11).

The invention relates to a medical hollow container, to such a medical hollow container having a closure cap, to a method for producing such a medical hollow container, and to a kit which comprises such a medical hollow container.

Medical hollow containers of the type discussed here are known. These generally cylindrically-shaped containers comprise a chamber for receiving the medical preparation, which chamber is delimited on both sides by a proximal and a distal end. The distal end, in particular with a distal opening, has a fastening structure, and the medical preparation can be removed from or introduced into the medicine container via the distal end. According to the design of the medicine container, individual distal closure devices for closing the distal end are arranged on the fastening structure, the distal closure device being adapted to the exact shape of the distal end of the given design of the medicine container. The proximal end is closed off by a portion of the inner wall of the base body, or has a proximal opening. The open proximal end is designed to receive a proximal closure device, in particular a stopper or a plunger. The plunger can be inserted into the proximal end, and the medical preparation can be expelled in the distal direction from the distal opening of the medical hollow container by a sliding movement of the plunger; or, in a reverse, proximal movement, a medical preparation can be drawn through this opening into the syringe.

Known medicine containers are typically in the form of a syringe, carpule, ampoule or injection bottle, i.e. a vial, can be made of glass or a plastics material, and typically have a base body with a central axis, the base body often being at least substantially rotationally symmetrical about the central axis. In this case, the base body encloses, at least in the circumferential direction around the central axis, a receiving portion which is designed to receive at least one medical preparation.

Syringes are closed in the direction of their proximal end with an axially, in particular distally and/or proximally displaceable closure device, which is typically designed as a plunger element operatively connectable to a plunger rod, in particular a sealing plunger element. By means of the plunger rod, it is possible, at least after the plunger rod has been operatively connected to the closing plunger element, to displace the closing plunger element in the distal and/or proximal direction and thereby dispense a medical preparation from the syringe and/or introduce it into the syringe.

Carpules, on the other hand, are closed with a stopper that does not protrude from the container, with no operative connection, in particular no screw connection, being provided between the stopper and a plunger rod. Instead, a separate dispensing device is provided for dispensing a medical preparation from a carpule, which at least partially penetrates the interior of the medicine container via the proximal opening and displaces the stopper into the base body of the carpule in such a way that the medical preparation is discharged at the distal end via a cannula which pierces a membrane-like closure.

Distal closure devices which have an injection device, in particular a cannula and/or a Luer lock fastening, can be fastened in particular to syringes. Vials, on the other hand, are not only intended for direct injection of a medical preparation, such that vials have a closure device at their distal end, which is designed, for example, as a stopper, cap or membrane closure. Similarly to vials, carpules typically have membrane-like closure devices which can be pierced with an injection needle or cannula.

A closure for a container for pharmaceutical preparations, and a container provided with this closure, are known from the German utility model DE 20 2018 102 680 U1. The closure for releasably closing an opening at the distal end of a cylindrical container for pharmaceutical preparations, in particular a syringe, preferably by means of a Luer lock fastening, comprises a cylindrical closure cap with a cylindrical cavity for receiving and holding a sealing element, the closure cap comprising an installation portion which points towards the container in the installed position, a sealing element with a sealing portion which is at least partially arranged inside the installation portion, and wherein the outer edge of the sealing element is set back from the outer edge of the installation portion. A device known as a Luer lock has become established in the medical field for fastening a cannula, for example, to a distal end of a container. In the case of containers made of plastic, this Luer lock attachment can be made as a single piece with the container. However, this is not possible in this way with medical hollow containers made of glass.

An apparatus for screwing media-carrying threaded connections, in particular Luer lock connections, is known from European patent EP 1 567 215 B1. The apparatus has an outer part and an inner part rotatably received therein, which has means for receiving the thread-bearing connecting part or an additional part secured against rotation to the same, wherein inhibiting means are present, which allow a rotation of the inner part relative to the outer part only in one direction of rotation, and oppose this rotation with a resistance.

German patent DE 603 14 018 T2 discloses a syringe arrangement for medical compositions, wherein at least one plunger element has a first stationary part, which has at least one flow passage, and a second displaceable part, which has at least one flow passage, wherein the first part and the second part are arranged side by side. In order to establish a flow connection between the two chambers, the flow passages are brought into alignment, in particular by turning. An anti-rotation function is provided in this case, wherein the first part of the plunger element preferably has an elliptical shape in order to provide such an anti-rotation function, and/or the first part of the plunger element has one or more grooves which can be brought into engagement with one or more corresponding ribs which are arranged on the inner surface of the syringe barrel. The first part is held in place as a result, while the second part can be rotated slightly.

The disadvantage of the prior art, however, is that when attaching a closure cap to a medical hollow container, in particular when rotating a closure cap onto a medical hollow container, the medical hollow container is at least partially rotated relative to the closure cap. As such, securing the closure cap on the medical hollow container is made more difficult.

The invention is based on the object of providing a medical hollow container, such a medical hollow container having a closure cap, a method for producing such a medical hollow container, and a kit which has such a medical hollow container, wherein the disadvantages mentioned do not occur and wherein in particular a rotation of the closure cap relative to the medical hollow container, in particular when the medical hollow container is closed by means of the closure cap, is at least largely prevented.

The object is achieved by creating the subject matter of the independent claims. Advantageous configurations are found in the dependent claims.

The object is achieved in particular by creating a medical hollow container, the medical hollow container being made of glass, wherein the medical hollow container has a fastening portion and a receiving portion, the fastening portion having a shoulder region on a side facing the receiving portion and having a head region on a side facing away from the receiving portion, and wherein the head region has an opening on which a closure cap can be arranged. The medical hollow container is characterized in that the fastening portion has at least one rotation inhibiting structure which is designed to at least inhibit relative rotation between the medical hollow container and the closure cap with respect to each other, wherein the rotation inhibiting structure has at least one rotation inhibiting element, wherein the at least one rotation inhibiting element is at least one elevation and/or at least one depression, and/or is designed as a non-circular shape of the opening and/or a non-circular edge of the opening.

The medical hollow container of the present invention has advantages compared to the prior art. Advantageously, particularly when the hollow container is closed with a closure cap, a relative rotation of the closure cap with respect to the medical hollow container is at least inhibited, which enables the medical hollow container to be more easily closed with the closure cap. Advantageously, a rotational movement of the closure cap relative to the medical hollow container is at least inhibited or is completely prevented, in particular when a medical apparatus is arranged and/or fastened to the closure cap, in particular the closure cap designed as a coupling element. Advantageously, the closure cap can be reversibly fixed, in particular closed, on the medical hollow container in a simple manner. Advantageously, the medical hollow container and the medical hollow container having the closure cap are particularly easy to handle.

A medical hollow container is understood to mean, in particular, a syringe, a carpule, an ampoule or a vial, the medical hollow container preferably being pre-fillable or pre-filled.

A depression is understood in particular to be an element which is suitable for allowing a further element, in particular an elevation or a projection, to be brought into engagement therein.

An elevation or a projection is understood in particular to be a protruding element, in particular an elongated element, which can engage in another element, in particular in a depression.

The medical hollow container is preferably designed as a cylindrical hollow container. The medical hollow container preferably has a radial inner dimension and a radial outer dimension, the radial inner dimension of the receiving portion preferably being the same over the entire axial length.

An axial direction is understood to mean, in particular, a direction which extends along the central axis or parallel to the central axis of the medical hollow container. A radial direction is perpendicular to the axial direction, and in particular to the central axis of the medical hollow container. A circumferential direction encompasses the axial direction.

The medical hollow container is preferably designed to receive a medical preparation.

A medical preparation, in particular a medicinal substance, is understood to mean, in particular, a single substance or a mixture of several substances, in particular a medicinal active substance and/or adjuvant, or a mixture of several such substances. The medical hollow container, in particular the receiving portion of the medical hollow container, is suitable for receiving the medical preparation and making it available for an injection and/or medical application.

A medical apparatus is understood to mean in particular an apparatus—preferably serving a medical and/or pharmaceutical purpose—which can be used with the medical hollow container, in particular connected to the medical hollow container, in particular connected in a manner allowing flow. The medical apparatus is preferably configured to receive or allow the passage of a fluid, in particular a liquid. In particular, the medical apparatus is a syringe, a tube, a connector, a connecting piece, an adapter, or an infusion container.

The medical hollow container, in particular the receiving portion of the medical hollow container, can preferably be sealed against contamination and/or germs in such a way that the receiving portion remains sterile after a prior sterilization.

The closure cap is preferably designed in such a way that the medical hollow container can be closed by the closure cap, in particular can be closed in a sterile manner. The closure cap is preferably constructed as a single piece; but alternatively, the closure cap can also preferably be constructed in several pieces.

The closure cap is preferably designed as a coupling part for connecting the medical hollow container to a further medical container, in particular a medical apparatus, for example a syringe.

The closure cap preferably has a holding element on the outside of the closure cap. This improves handling by the user. The holding element preferably has at least one soft component, in particular for the purpose of improved gripping by a user, at least one elevation or depression, in particular at least one rib, at least one nub, and/or a corrugated structure, preferably a plurality of elevations and/or depressions, preferably a plurality of ribs and/or nubs.

The sealing element is preferably designed as a single piece. Alternatively, however, the sealing element can also be composed of several pieces.

The sealing element preferably has at least largely the shape of the cavity between the closure cap and the fastening portion of the medical hollow container. As a result, the sealing element can seal the cavity particularly well.

The sealing element is preferably formed from an elastic, in particular elastomeric, material.

The closure cap preferably has a wall thickness at the thickest point of at most 2.5 mm, preferably of at most 2 mm, wherein the thickness of the wall can be designed differently over the entire length of the closure cap.

The rotation inhibiting element preferably has a width of less than 1 mm, preferably less than 0.9 mm, preferably less than 0.8 mm, preferably less than 0.7 mm, preferably less than 0.6 mm, preferably less than 0.5 mm, or preferably less than 0.4 mm.

According to a further development of the invention, it is provided that the at least one rotation inhibiting element is at least one elevation, the at least one elevation being roof-shaped, pyramidal, tetrahedral, cylindrical or hemispherical. If the elevation has edges, these are preferably chamfered and/or interrupted. The geometries proposed here have proven to be particularly suitable as shapes for rotation inhibiting elements.

According to a further development of the invention, it is provided that the rotation inhibiting structure has a plurality of rotation inhibiting elements, the rotation inhibiting elements being arranged, preferably molded, in the circumferential direction around the central axis of the medical hollow container.

The rotation inhibiting structure preferably has at least two rotation inhibiting elements, preferably at least three, preferably at least four, preferably at least six, preferably at least eight, preferably at least ten, preferably at least twenty, or preferably more than twenty rotation inhibiting elements.

The rotation inhibiting elements are preferably arranged, preferably molded, symmetrically in the circumferential direction around the central axis of the medical hollow container. The rotation inhibiting structure preferably has a plurality of mutually identical rotation inhibiting elements.

The rotation inhibiting elements are preferably designed as ribs.

In an alternative embodiment, the rotation inhibiting structure has differently designed rotation inhibiting elements. This ensures a particularly good anti-rotation protection.

According to a further development of the invention, it is provided that the non-circular shape of the opening and/or the non-circular edge is elliptical, polygonal, jagged, or grooved. This ensures a particularly good anti-rotation protection.

According to a further development of the invention, it is provided that the medical hollow container is a carpule, a syringe or a vial.

The sealing element is preferably held fixed in the closure cap, such that the sealing element cannot be rotated relative to the closure cap. The sealing element is preferably glued to the inside of the closure cap.

A fastening portion is understood to mean, in particular, a portion facing the distal end of the medical hollow container, the fastening portion at least partially having a rotation inhibiting structure. The fastening portion is preferably arranged in the region of the medical hollow container in which a closure cap can be arranged and/or a distal closure device is provided for closing the medical hollow container, and/or is secured against rotation by means of the rotation inhibiting structure.

The fastening portion is preferably arranged on the inside and/or outside of the shoulder region.

The fastening portion is preferably arranged on the inside and/or outside of the head region.

The medical hollow container preferably has a radial projection for fastening a closure cap, in particular on the outer wall of the medical hollow container, wherein a fastening structure is able to be grasped from behind by the closure cap. In particular, a fastening structure is thus provided for the closure cap, which closure cap can snap into place into the same behind the radial projection. The projection itself preferably has at least one radial recess, in particular a radial annular groove, into which the closure cap can snap into place in a further locking position.

The projection of the fastening structure, in particular the fastening structure itself, is preferably formed as a single piece together with the medical hollow container.

The object is also achieved by creating a medical hollow container according to the invention, having a closure cap arranged on the fastening portion of the medical hollow container, in particular according to one of the embodiments described above. The advantages that have already been explained in connection with the medical hollow container apply in particular for the medical hollow container having the closure cap. The closure cap is arranged on a side of the fastening portion of the medical hollow container facing away from the receiving portion, wherein the medical hollow container can be closed, and preferably is closed, by the closure cap, wherein a cavity is formed in the region between the closure cap and the fastening portion of the medical hollow container, in which a sealing element is at least partially arranged. The medical hollow container having the closure cap is characterized in that the fastening portion of the medical hollow container has at least one rotation inhibiting structure on the outside, wherein the closure cap has at least one rotation inhibiting element on the inside, wherein the at least one rotation inhibiting element of the closure cap is designed to be complementary to the at least one rotation inhibiting element of the medical hollow container, wherein the rotation inhibiting element of the closure cap lies opposite the rotation inhibiting element of the fastening portion of the medical hollow container in a closed position, wherein the fastening portion of the medical hollow container having the closure cap forms a non-positive, materially-bonded, and/or positive connection, such that a rotation of the closure cap relative to the fastening portion is at least inhibited, wherein the closure cap preferably is not rotatable relative to the fastening portion. Because the at least one rotation inhibiting element of the closure cap is complementary to the at least one rotation inhibiting element of the medical hollow container, the at least one rotation inhibiting element of the closure cap can be brought into engagement with the at least one rotation inhibiting element of the medical hollow container. Such engagement prevents rotation of the at least one rotation inhibiting element of the closure cap relative to the at least one rotation inhibiting element of the medical hollow container.

The sealing element is preferably designed as a single piece. Alternatively, however, the sealing element can also be composed of several pieces.

The sealing element preferably has at least largely the shape of the cavity between the closure cap and the fastening portion of the medical hollow container. As a result, the sealing element can seal the cavity particularly well.

The sealing element is preferably formed from an elastic, in particular elastomeric, material.

The sealing element is preferably held fixed in the closure cap, such that the sealing element cannot be rotated relative to the closure cap. The sealing element is preferably glued to the inside of the closure cap.

The sealing element is preferably formed over the entire cavity between the closure cap and the fastening portion of the medical hollow container; in particular, the sealing element fills the entire cavity. According to a further preferred embodiment, the sealing element only partially fills the cavity.

A distal closure device in the form of a closure cap, by means of which the medical hollow container can be closed in the distal direction, is preferably arranged at the opening of the medical hollow container. The closure cap can preferably be at least partially inserted reversibly into the medical hollow container, and in particular designed as a stopper, membrane closure or injection device. The closure cap is preferably a closure cap which is closed and which is continuous for, and in particular can be penetrated by, a cannula.

The closure cap is preferably secured against rotation relative to the sealing element in the region of the fastening portion of the medical hollow container by at least one rotation inhibiting element on the inner wall of the closure cap, in particular via at least one elongated rotation inhibiting element which is oriented in its main direction and which protrudes radially inward. In a preferred embodiment, at least two such rotation inhibiting elements are preferably provided.

The closure cap preferably has at least one rotation inhibiting element on the inside, preferably at least two, preferably at least four, preferably at least six, or preferably at least ten rotation inhibiting elements on the inside.

The rotation inhibiting elements are preferably arranged uniformly on the inside of the closure cap in the circumferential direction, in particular at equal intervals. The at least one rotation inhibiting element on the inside of the closure cap can counteract rotation of the sealing element relative to the closure cap, for example during the installation, i.e., the coupling, of a medical apparatus to the closure cap, in particular to the closure cap designed as a coupling element.

The object is also achieved by creating a medical hollow container according to the invention, having a closure cap arranged on the fastening portion of the medical hollow container, in particular according to one of the embodiments described above. The advantages that have already been explained in connection with the medical hollow container apply in particular for the medical hollow container having the closure cap. In a further embodiment, the closure cap is arranged on the medical hollow container having the closure cap on a side of the fastening portion of the medical hollow container facing away from the receiving portion, wherein the medical hollow container can be closed by the closure cap, wherein a cavity in which a sealing element is at least partially arranged is formed in the region between the closure cap and the fastening portion of the medical hollow container. The medical hollow container having the closure cap is characterized in that the fastening portion of the medical hollow container has at least one rotation inhibiting element on the inside, wherein the sealing element is flexible and wherein the sealing element engages or is molded into the at least one rotation inhibiting element of the fastening portion, such that the sealing element and the medical hollow container cannot be rotated relative to each other.

According to a further development of the invention, it is provided that the sealing element is fixed at least in some portions thereof relative to the closure cap, and in particular forms a non-positive, materially-bonded, and/or positive connection, such that the closure cap and the sealing element cannot be rotated relative to each other. In particular, it is possible for the sealing element to interact with the closure cap via a rotation inhibiting structure, in particular according to any of the configurations described above, a non-positive fit, or an adhesive connection, such that the closure cap and the sealing element cannot be rotated relative to each other.

According to a development of the invention, it is provided that the sealing element is made of an elastic material.

The shape and/or the size of the rotation inhibiting element on the inside of the closure cap are preferably adapted to the elastic and/or elastomeric properties of the sealing element.

According to a further development of the invention, it is provided that the closure cap is designed as a coupling element or has a coupling element, wherein the coupling element is designed for axially connecting the medical hollow container to a medical apparatus, in particular in a manner allowing flow, in particular for producing a flow channel between the medical hollow container and the medical apparatus, in particular by means of a Luer lock connection.

Luer lock connections are used in particular in the field of medical technology in order to connect parts to each other, in the interior of which a medium, in particular a liquid, will flow. These parts include, for example, infusion containers and associated lines or injection syringes and associated needles. To ensure that such connections are tight and do not loosen, they should preferably be tightened to a certain torque.

The medical apparatus is preferably a syringe, a tube or an infusion container.

The closure cap is preferably made from a thermoplastic material.

The object is also achieved by creating a method for producing a medical hollow container according to the invention in a rotary process, in particular a medical hollow container according to any of the previously described embodiments, wherein a rotating glass tube is heated, wherein the receiving portion and the fastening portion with the head region and the shoulder region are shaped by means of forming rollers, such that a molded glass container is obtained. The method is particularly characterized in that the at least one elevation and/or the at least one depression are formed from the molded glass container or are formed into the molded glass container, and/or the opening itself and/or the edge of the opening is not round. For the method for producing a medical hollow container, this results particularly in the advantages that have already been explained in connection with the medical hollow container.

According to a further development of the invention, it is provided that to form the at least one elevation, the molded glass container is partially heated, wherein a mold for forming the at least one elevation is applied to the molded glass container from the outside, and wherein a pressure medium inside the hollow container presses the molded, partially heated glass container against the mold, such that the at least one elevation is formed in the molded glass container.

The pressure medium is preferably a liquid or gaseous medium.

According to a further development of the invention, it is provided that the molded glass container is partially heated to form the at least one depression, wherein a mold for forming the at least one depression is applied from the outside and/or from the inside to the molded, partially heated glass container, and wherein, the mold is pressed against the molded, partially heated glass container by means of pressure, such that the at least one depression is formed in the molded glass container.

According to a further development of the invention, it is provided that a non-circular pin is arranged in the opening in the head region of the molded glass container to form the non-circular shape of the opening when the shape of the head region is rolled by means of forming rollers, wherein the non-circular pin is rotated at the same speed as the glass tube being formed, such that the non-circular shape of the opening is formed by means of the non-circular pin.

The invention also relates to a medical hollow container according to the invention, or to a medical hollow container according to any of the embodiments described above, in combination with a medical apparatus, the medical hollow container being connected to the medical arrangement, in particular connected in a manner allowing flow. In particular, a flow channel is formed between the medical hollow container and the medical apparatus, preferably via the closure cap configured as a coupling element.

The invention also relates to a kit for medical use, comprising a medical hollow container according to the invention, or a medical hollow container according to any of the embodiments described above, wherein the medical hollow container is present in sterile packaging, wherein the medical hollow container is preferably filled with at least one medical preparation, and wherein the kit preferably comprises instructions for use for the medical hollow container and/or an instruction leaflet.

The kit preferably comprises a medical apparatus for connection, in particular in a manner allowing flow, to the medical hollow container.

The invention is explained in greater detail below with reference to the drawing. In the drawing:

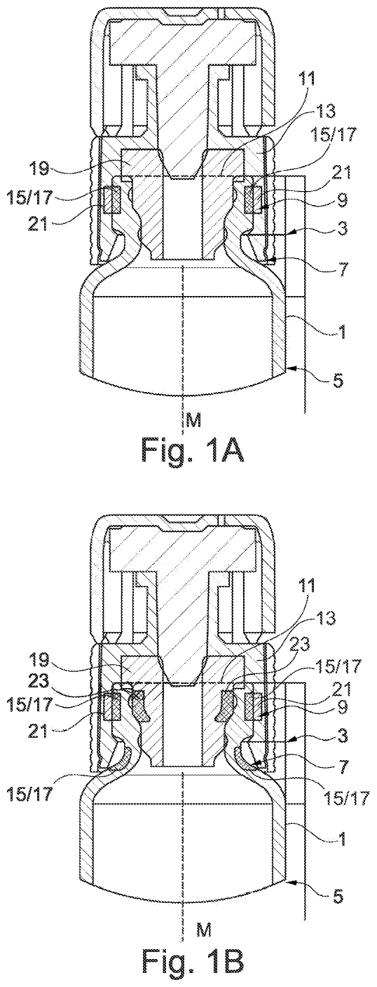

FIG. 1 is a schematic representation of a medical hollow container having a closure cap in a side view, in several embodiments (FIGS. 1A to 1C);

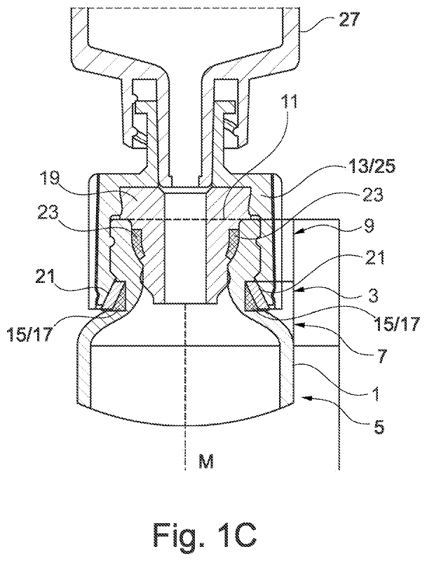

FIG. 2 is a schematic representation of a medical hollow container in the form of a syringe with a closure cap (FIG. 2A) and of a medical hollow container in the form of a syringe having a closure cap with a coupling element with a connected medical apparatus (FIG. 2B), in one embodiment; and

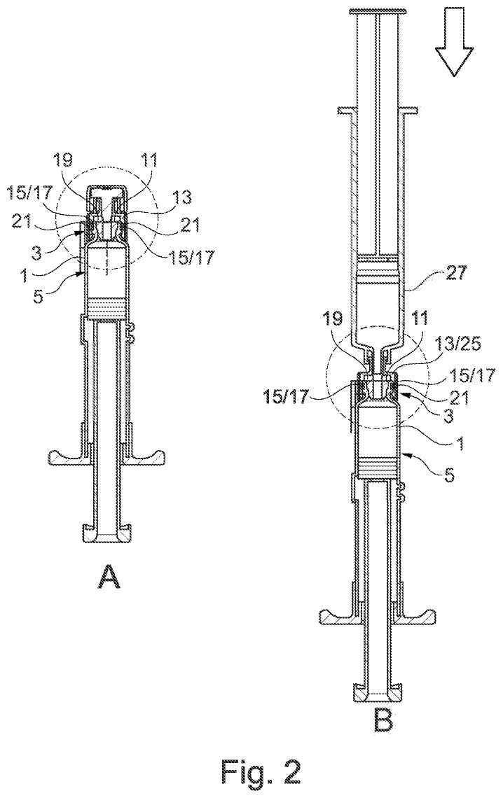

FIG. 3 is a schematic representation of medical hollow containers in various embodiments (FIG. 3A to 3D).

FIG. 1 shows a schematic representation of a medical hollow container 1 having a closure cap 13 in several embodiments (FIGS. 1A, 1B and 1C).

The medical hollow container 1 is made of glass, and the medical hollow container 1 has a fastening portion 3 and a receiving portion 5, wherein the fastening portion 3 has a shoulder region 7 on a side facing the receiving portion 5 and a head region 9 on a side facing away from the receiving portion 5, and wherein the head region 9 has an opening 11 on which a closure cap 13 can be arranged. The fastening portion 3 has at least one rotation inhibiting structure 15 which is designed to at least inhibit relative rotation between the medical hollow container 1 and the closure cap 13 with respect to each other, the rotation inhibiting structure 15 having at least one rotation inhibiting element 17, the at least one rotation inhibiting element 17 being at least one elevation and/or at least one depression, and/or being designed as a non-circular shape of the opening 11 and/or a non-circular edge of the opening 11.

When the medical hollow container 1 is closed with the closure cap 13, the latter interacts—indirectly or directly—with the rotation inhibiting structure 15, thereby at least preventing a rotation of the closure cap 13 relative to the medical hollow container 1, which makes it easier to close the medical hollow container 1 with the closure cap 13. A relative rotational movement of the closure cap 13 with respect to the medical hollow container 1 is at least inhibited or is completely prevented, in particular when a medical apparatus 27 is arranged on or fastened to the closure cap 13, in particular the closure cap 13 designed as a coupling element 25. The medical hollow container 1 and the medical hollow container 1 having the closure cap 13 are particularly easy to handle.

In one embodiment of the invention, the rotation inhibiting structure 15 has a plurality of rotation inhibiting elements 17, the rotation inhibiting elements 17 being arranged, preferably molded, in the circumferential direction around the central axis M of the medical hollow container 1.

In a further embodiment of the invention, the non-circular shape of the opening 11 and/or the non-circular edge is elliptical, polygonal, jagged or grooved.

In a further embodiment of the invention, the medical hollow container 1 is a carpule, a syringe or a vial.

In one embodiment, the medical hollow container 1 has a closure cap 13 arranged on the fastening portion 3 of the medical hollow container 1, the closure cap 13 being arranged on a side of the fastening portion 3 of the medical hollow container 1 facing away from the receiving portion 5, wherein the medical hollow container 1 can be closed by the closure cap 13, wherein a cavity is formed in the region between the closure cap 13 and the fastening portion 3 of the medical hollow container 1, in which cavity a sealing element 19 is at least partially arranged. The fastening portion 3 of the medical hollow container 1 has at least one rotation inhibiting structure 15 on the outside, wherein the closure cap 13 has at least one rotation inhibiting element 21 on the inside, wherein the at least one rotation inhibiting element 21 of the closure cap 13 is designed to be complementary to the at least one rotation inhibiting element 17 of the medical hollow container 1. The rotation inhibiting element 21 of the closure cap 13 lies opposite the rotation inhibiting element 17 of the fastening portion 3 of the medical hollow container 1 in a closed position, the fastening portion 3 of the medical hollow container 1 forming a non-positive, materially-bonded, and/or positive connection with the closing cap 13, such that the closure cap 13 cannot be rotated relative to the fastening portion 3 (FIG. 1A).

In an alternative embodiment, the medical hollow container 1 has a closing cap 13 arranged on the fastening portion 3 of the medical hollow container 1, the closing cap 13 being arranged on the side of the fastening portion 3 of the medical hollow container 1 facing away from the receiving portion 5, wherein the medical hollow container 1 can be closed by the closure cap 13, wherein in the region between the closure cap 13 and the fastening portion 3 of the medical hollow container 1, a cavity is formed in which a sealing element 19 is at least partially arranged. The fastening portion 3 of the medical hollow container 1 has at least one rotation inhibiting element 23 on the inside, wherein the sealing element 19 is flexible, the sealing element 19 engaging in the at least one rotation inhibiting element 23 of the fastening portion 3, such that the sealing element 19 and the medical hollow container 1 are not rotatable relative to each other (see FIGS. 1B and 1C).

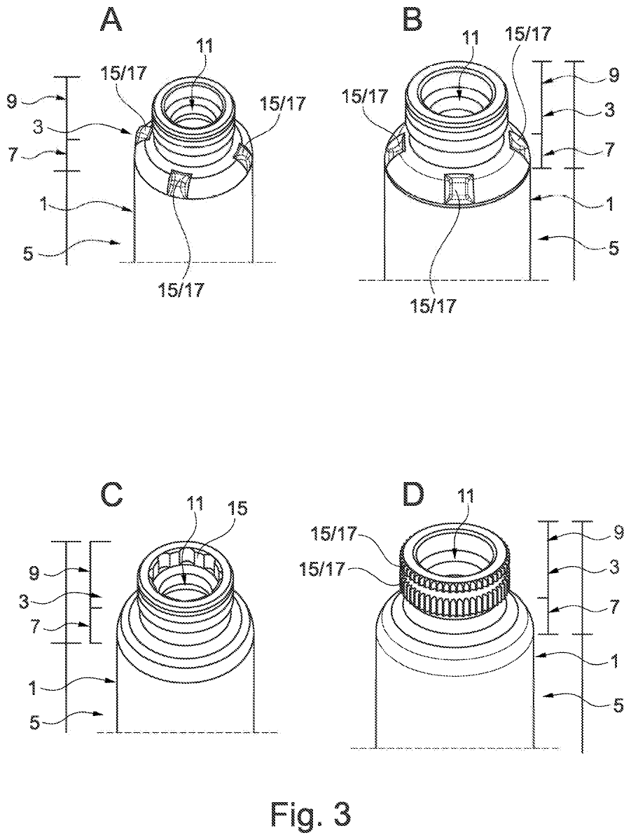

In FIGS. 1B and 1C, in one embodiment of the invention, the medical hollow container 1 has at least one rotation inhibiting element 23 on the inside and at least one rotation inhibiting structure 15 on the outside. The rotation inhibiting structure 15 is designed to be complementary to at least one rotation inhibiting element 21 on the inside of the closure cap 13. In FIG. 1B the rotation inhibiting structure 15 and the rotation inhibiting element 21 are formed in the head region 9 of the fastening portion 3, and in FIG. 10 the rotation inhibiting structure 15 and the rotation inhibiting element 21 are constructed in the shoulder region 7 of the fastening portion 3. In a further embodiment, it is conceivable that at least one rotation inhibiting structure 15 and one rotation inhibiting element 21 of the closure cap 13 are formed both in the head region 9 and in the shoulder region 7.

In a further embodiment of the invention, the sealing element 19 is fixed at least in some portions thereof relative to the closure cap 13, in particular by forming a non-positive, materially-bonded, and/or positive connection with it, such that the closure cap 13 and the sealing element 19 cannot be rotated relative to each other.

In a further embodiment of the invention, the sealing element 19 is made of an elastic material.

In a further embodiment of the invention, the rotation inhibiting structure 15 has—preferably exclusively—the at least one inside rotation inhibiting element 23 in the fastening portion 3 in which the sealing element 19 engages. The sealing element 19 is preferably fixed at least in some portions thereof relative to the closure cap 13, in particular by forming a non-positive, materially-bonded, and/or positive connection to it, such that the closure cap 13 and the sealing element 19 cannot be rotated relative to each other. In a preferred embodiment, the sealing element 19 and the closure cap 13 are connected to each other non-positively.

Alternatively or additionally, the sealing element 19 and the closure cap 13 are connected to each other by at least one additional rotation inhibiting element 17, 21.

Alternatively or additionally, the sealing element 19 and the closure cap 13 are glued to each other.

In a further embodiment of the invention, the closure cap 13 is designed as a coupling element 25, the coupling element 25 being designed for axially connecting the medical hollow container 1 to a medical apparatus 27 (FIG. 2B), in particular by means of a Luer lock connection.

FIG. 2 is a schematic representation of a medical hollow container 1 in the form of a carpule having a closure cap 13 (FIG. 2A), and of a medical hollow container 1 in the form of a carpule having a closure cap 13 with a coupling element 25 with a connected medical apparatus 27 (FIG. 2B), in one embodiment. Identical and functionally identical elements are provided with the same reference symbols, such that in this respect reference is made to the preceding description. In the present embodiment, the medical apparatus 27 is a syringe.

FIG. 3 is a schematic representation of a medical hollow container 1 in various configurations (FIGS. 3A, 3B, 3C and 3D). Identical and functionally identical elements are provided with the same reference symbols, such that in this respect reference is made to the preceding description.

In FIG. 3A, the medical hollow container 1 has, in one embodiment, a rotation inhibiting structure 15 in the shoulder region 7 of the fastening portion 3, the rotation inhibiting structure 15 having a plurality of rotation inhibiting elements 17, the rotation inhibiting elements 17 being shaped as elevations.

In a preferred embodiment, the elevations are roof-shaped, in particular chamfered or with interrupted edges. The elevations can also be configured differently, preferably to be pyramidal, tetrahedral, cylindrical, or hemispherical, in each case—if there are edges on the geometry—particularly preferably chamfered or with interrupted edges.

In FIG. 3B, the medical hollow container 1 has, in one embodiment, in the shoulder region 7 of the fastening portion 3, a rotation inhibiting structure 15 with a plurality of rotation inhibiting elements 17, the rotation inhibiting elements 17 being designed as depressions. In FIG. 3C, in one configuration, the medical hollow container 1 has a rotation inhibiting structure 15 on the inside of the fastening portion 3, the rotation inhibiting structure 15 being grooved. In FIG. 3D, the medical hollow container 1 has, in one embodiment, a rotation inhibiting structure 15 in the head region 9 of the fastening portion 3, the rotation inhibiting structure 15 being formed from a plurality of rotation inhibiting elements 17 which are arranged distributed along the circumferential direction of the medical hollow container 1, in particular in the form of a knurling.

The medical hollow container 1 is produced in a rotary process. A rotating glass tube is heated, the receiving portion 5 and the fastening portion 3 with the head region 9 and the shoulder region 7 being shaped by means of forming rollers, such that a molded glass container is obtained. The at least one elevation and/or the at least one depression are molded from the molded glass container or molded into the molded glass container, and/or the opening 11 itself and/or the edge of the opening 11 are non-circular.

In one embodiment of the method of the invention, the molded glass container is partially heated to form the at least one elevation, a mold for forming the at least one elevation being applied to the molded glass container from the outside, wherein the molded, partially heated glass container is pressed against the mold by means of a pressure medium, in particular a gas or a liquid, in the interior of the hollow container, such that the at least one elevation is formed in the molded glass container.

In a further embodiment of the method of the invention, the molded glass container is partially heated to form the at least one depression, a mold for forming the at least one depression being applied to the partially heated glass container from the outside and/or from the inside, and by means of pressure the mold is pressed against the molded, partially heated glass container, such that the at least one depression is formed in the molded glass container.

In a further embodiment of the method of the invention, to form the non-circular shape of the opening 11, a non-circular pin is arranged in the opening 11 in the head region 9 of the molded glass container when the shape of the head region 9 is rolled by means of forming rollers, wherein the non-circular pin is rotated at the same speed as the glass tube being formed, such that the non-circular opening 11 is formed by means of the non-circular pin.

1. A medical hollow container (1), wherein the medical hollow container (1) is made of glass, wherein the medical hollow container (1) has a fastening portion (3) and a receiving portion (5), wherein the fastening portion (3) has a shoulder region (7) on a side facing the receiving portion (5) and has a head region (9) on a side facing away from the receiving portion (5), and wherein the head region (9) has an opening (11) on which a closure cap (13) can be arranged, characterized in that the fastening portion (3) has at least one rotation inhibiting structure (15) which is designed to at least inhibit relative rotation between the medical hollow container (1) and the closure cap (13) with respect to each other, wherein the rotation inhibiting structure (15) has at least one rotation inhibiting element (17), wherein the at least one rotation inhibiting element (17) is at least one elevation and/or at least one depression, and/or is designed as a non-circular shape of the opening (11) and/or a non-circular edge of the opening (11).

2. The medical hollow container (1) according to claim 1, characterized in that the at least one rotation inhibiting element (17) is at least one elevation, wherein the at least one elevation has a geometric shape selected from a group consisting of: a roof shape, a pyramidal shape, a tetrahedral shape, a cylindrical shape, and a hemispherical shape.

3. The medical hollow container (1) according to claim 1, characterized in that the rotation inhibiting structure (15) has several rotation inhibiting elements (17), the rotation inhibiting elements (17) being arranged, preferably molded, in the circumferential direction around the central axis of the medical hollow container (1).

4. The medical hollow container (1) according to claim 1, characterized in that the non-circular shape of the opening (11) and/or the non-circular edge is elliptical, polygonal, jagged, or grooved.

5. The medical hollow container (1) according to claim 1, characterized in that the medical hollow container (1) is a carpule, a syringe or a vial.

6. The medical hollow container (1) according to claim 1, having a closure cap (13) arranged on the fastening portion (3) of the medical hollow container (1), the closure cap (13) being arranged on a side of the fastening portion (3) facing away from the receiving portion (5) of the medical hollow container (1), wherein the medical hollow container (1) can be closed by the closure cap (13), wherein a cavity is formed in the region between the closure cap (13) and the fastening portion (3) of the medical hollow container (1), in which cavity a sealing element (19) is at least partially arranged, characterized in that the fastening portion (3) of the medical hollow container (1) has the at least one rotation inhibiting structure (15) on the outside, the closure cap (13) having at least one rotation inhibiting element (21) on the inside, wherein the at least one rotation inhibiting element (21) of the closure cap (13) is designed to be complementary to the at least one rotation inhibiting element (17) of the medical hollow container (1), wherein the rotation inhibiting element (21) of the closure cap (13) in a closed position is opposite the rotation inhibiting element (17) of the fastening portion (3) of the medical hollow container (1), the fastening portion (3) the medical hollow container (1) forming a non-positive, materially-bonded, and/or positive connection with the closure cap (13), such that the closure cap (13) cannot be rotated relative to the fastening portion (3).

7. The medical hollow container (1) according to claim 1, having a closure cap (13) arranged on the fastening portion (3) of the medical hollow container (1), the closure cap (13) being arranged on the side of the fastening portion (3) facing away from the receiving portion (5) of the medical hollow container (1), wherein the medical hollow container (1) can be closed by the closure cap (13), wherein a cavity is formed in the region between the closure cap (13) and the fastening portion (3) of the medical hollow container (1), in which cavity a sealing element (19) is at least partially arranged, characterized in that the fastening portion (3) of the medical hollow container (1) has at least one rotation inhibiting element (23) on the inside, and the sealing element (19) is flexible, wherein the sealing element (19) engages in the at least one rotation inhibiting element (23) of the fastening portion (3), such that the sealing element (19) and the medical hollow container (1) cannot be rotated relative to each other.

8. The medical hollow container (1) having a closure cap (13) according to claim 6, characterized in that the sealing element (19) is fixed at least in portions thereof relative to the closure cap (13), and in particular forms a non-positive, materially-bonded, and/or positive connection, such that the closure cap (13) and the sealing element (19) cannot be rotated relative to each other.

9. The medical hollow container (1) having a closure cap (13) according to claim 6, characterized in that the sealing element (19) is made of elastic material.

10. The medical hollow container (1) having a closure cap (13) according to claim 6, characterized in that the closure cap (13) is designed as a coupling element (25), the coupling element (25) being designed for axially connecting the medical hollow container (1) to a medical apparatus (27), in particular by means of a Luer lock connection.

11. A method for producing a medical hollow container (1) according to claim 1 in a rotary process, wherein a rotating glass tube is heated, wherein the receiving portion (5) and the fastening portion (3) with the head region (9) and the shoulder region (7) are shaped by means of forming rollers, such that a molded glass container is obtained, characterized in that the at least one elevation and/or the at least one depression is/are molded from the molded glass container or molded into the molded glass container, and/or the opening (11) and/or the edge of the opening (11) are not round.

12. The method according to claim 11, characterized in that, to form the at least one elevation, the molded glass container is partially heated, a mold for forming the at least one elevation being applied from the outside to the shaped, partially heated glass container, and a pressure medium inside the hollow container pressing the molded, partially heated glass container against the mold, such that the at least one elevation is formed in the molded glass container.

13. The method according to claim 11, characterized in that the molded glass container is partially heated to form the at least one depression, a mold for forming the at least one depression being applied from the outside and/or from the inside to the molded, partially heated glass container, and the mold being pressed against the molded, partially heated glass container by means of pressure, such that the at least one depression is formed in the molded glass container.

14. The method according to claim 11, characterized in that, in order to form the non-circular shape of the opening (11), a non-circular pin is arranged in the opening in the head region of the molded glass container when the shape of the head region is rolled by means of forming rollers, wherein the non-circular pin is rotated at the same speed as the glass tube being formed, such that the non-circular shape of the opening (11) is formed by means of the non-circular pin.

15. A medical hollow container (1) according to claim 1, in combination with a medical apparatus (27), wherein the medical hollow container (1) is connected, in particular in a manner allowing flow, to the medical arrangement (27).

16. A kit for medical use, comprising a medical hollow container (1) according to claim 1, wherein the medical hollow container (1) is present in sterile packaging, the medical hollow container (1) preferably being filled with at least one medical preparation, and the kit preferably comprising instructions for use for the medical hollow container (1) and/or an instruction leaflet.
